(19) United States
(12) Patent Application Publication
(10) Pub. No.: US 2005/0226365 A1
(43) Pub. Date: Oct. 13, 2005

(54) Radius-in-image dependent detector row filtering for windmill artifact reduction

(75) Inventor: Katsuyuki Taguchi, Buffalo Grove, IL (US)

Correspondence Address: OBLON, SPIVAK, MCCLELLAND, MAIER & NEUSTADT, P.C., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-Shi (JP)

(21) Appl. No.: 10/812,187

(22) Filed: Mar. 30, 2004

(51) Int. Cl. G21K 1/12; H05G 1/60; A61B 6/00; G01N 23/00

(52) U.S. Cl. 378/13

(57) ABSTRACT

A method, X-ray CT apparatus, and computer program product for obtaining data from a computed tomography (CT) scan, wherein projection data are obtained from at least two detector rows in a CT system, the projection data are filtered in a direction of the at least two detector rows to obtain filtered data in which windmill artifacts are reduced, and the filtered data are reconstructed.

### BACKGROUND OF THE INVENTION

1. Field of the Invention

Multi-slice x-ray computer tomography (CT) systems were developed and introduced into the medical market around 1998. The number of slices generally ranges from 2 to 40 currently, and is expected to increase to 64 or even 256. (See Y. Saito, H. Aradate, H. Miyazaki, K. Igarashi, and H. Ide, “Development of a Large Area 2-dimensional Detector for Real-Time 3-dimensional CT (4D-CT),” Radiology vol. 217 (P), 405 (2000); Y. Saito, H. Aradate, H. Miyazaki, K. Igarashi, and H. Ide, “Large Area Two-Dimensional Detector System for Real-Time Three-Dimensional CT (4D-CT),” Proc. of SPIE Med. Imag. Conf., 4320, 775-782 (2001); and http://www3.toshiba.co.jp/medical/4d-ct/, the contents of each of which are herein incorporated by reference). One cone-beam image reconstruction algorithm with helical scanning for CT systems uses a generalized weighted version of Feldkamp reconstruction algorithm, which introduces a flexible focus orbit and applies a weighting function to the Feldkamp algorithm. (See L. A. Feldkamp, L. C. Davis, and J. W. Kress, “Practical Cone-Beam Algorithm,” J. Opt. Soc. Am. A, 6, 612-19 (1984); H. Aradate and K. Nambu, “Computed Tomography Apparatus,” Japanese Patent No. 2,825,352; L. G. Zeng and G. T. Gullberg, “Short-scan Cone Beam Algorithm for Circular and Noncircular Detector Orbit,” Proc. of SPIE Med. Imag. Conf, 1233, 453-463 (1990); H. Kudo and T. Saito, “Three-Dimensional Helical-Scan Computed Tomography Using Cone-Beam Projections,” J. Electron. Information Commun. Soc. Japan, J74-D-II, 1108-1114 (1991); G. Wang, T. H. Lin, P. C. Cheng, D. M. Shinozaki, “A General Cone-Beam Reconstruction Algorithm,” IEEE Trans. Med. Imaging, 12, 486-496 (1993); K. Taguchi, “X-ray Computed Tomography Apparatus,” U.S. Pat. No. 5,825,842 (Filed in 1995); K. Wiesent, K. Barth, N. Novab, et al., “Enhanced 3-D-Reconstruction Algorithm for C-arm Systems Suitable for Interventional Procedures,” IEEE Trans. Med. Imaging, 19, 391-403 (2000); M. D. Silver, K. Taguchi, and K. S. Han, “Field-of-View Dependent Helical Pitch in Multi-Slice CT,” Proc. of SPIE Med. Imag. Conf, 4320, 839-850 (2001); M. D. Silver, K. Taguchi, and I. A. Hein, “A Simple Algorithm for Increased Helical Pitch in Cone-Beam CT,” The Sixth International Meeting on Fully Three-Dimensional Image Reconstruction in Radiology and Nuclear Medicine, 70-73 (2001), the contents of each are herein incorporated by reference).

Other reconstruction algorithms include quasi-cone-beam algorithms such as advanced single-slice rebinning (ASSR) and adaptive multiple plane reconstruction (AMPR), which reconstruct multiple slices that are not aligned to a single axis. The axes of the slices fit a helical orbit and may be z-filtered to obtain perpendicular slices. (See Y. Machida, “Computed Tomography System,” Japanese Patent Disclosure (Kokai) 08-187240; M. Kachelriess, S. Schaller, W. A. Kalender, “Advanced Single-slice Rebinning in Cone-Beam Spiral CT,” Medical Physics vol. 27, pp. 754-772 (2000); S. Schaller, K. Stierstorfer, H. Bruder, M. Kachelriess, and T. Flohr, “Novel Approximate Approach for High-Quality Image Reconstruction in Helical Cone Beam CT at Arbitrary Pitch,” Proc of SPIE Vol. 4322, pp. 113-127 (2001); T. Flohr, K. Stierstorfer, H. Bruder, J. Simon, A. Polacin, and S. Schaller, “Image Reconstruction and Image Quality Evaluation for a 16-slice CT Scanner,” Medical Physics vol. 30, pp. 832-845 (2003), the contents of all of which are herein incorporated by reference).

There are some other algorithms which have the same aliasing problem by using cone-to-parallel fan-beam rebinning (See, e.g., H. Tuy, “3D Image Reconstruction for Helical Partial Cone-beam Scanners,” proc of Fully 3D 1999, pp. 7-10; H. Turbell, et al., “Three-dimensional Image Reconstruction in Circular and Helical Computed Tomography,” Licentiate thesis No. 760, Linkoping Univ, ISBN 91-7219-463-4, 1999; H. Turbell, et al., “An Improved PI-method for Reconstruction from Helical Cone-beam Projections,” Conf record of IEEE MIC 1999; R. Manzke et al, “Extended Cardiac Reconstruction (ECR): A Helical Cardiac Cone-beam Reconstruction Method,” proc of Fully 3D 2003, Mo-PM2-4., the contents of all of which are herein incorporated by reference), and fan-beam algorithms (K. Taguchi and H. Anno, “High Temporal Resolution for Multi-slice Helical CT,” Medical Physics vol. 27, May 2000, the contents of which are herein incorporated by reference). The present invention is not limited by the choice of reconstruction scheme.

2. Description of the Related Art

One common problem with cone beam or quasi-cone-beam algorithms is insufficient sampling intervals in the z-axis (detector row) direction. These algorithms violate the Nyquist theorem (which requires two samples within one detector cell aperture) and causes aliasing artifacts in reconstructed images due to high frequency components. (See M. Silver, K. Taguchi, I. Hein, B. Chiang, M. Kazama, I. Mori, “Windmill Artifact in Multi-Slice Helical CT,” Proc of SPIE Vol. 5032, pp. 1918-1927 (2003), the contents of which are herein incorporated by reference). These aliasing artifacts are known as windmill artifacts. Presently, there are two types of methods to overcome this problem. One method changes hardware, and the other method alters software.

The hardware modification applies a flying focus technique (in xy-plane, detector channel direction) to the z-axis direction and combines two projection sets with N samples in z into one set of two N samples in z. (See T. Flohr, H. K. Bruder, K. Stierstorfer, S. Schaller, “Evaluation of Approaches to Reduce Spiral Artifacts in Multi-Slice Spiral CT,” RSNA 2003 program, pp 567, the contents of which are herein incorporated by reference.) However, this potential solution has the following disadvantages: (1) the data size becomes twice as large as currently used, (2) the scanners become more expensive, and (3) a new image reconstruction algorithm is required, among other things.

The software solution may include non-adaptive z-filtering to reduce z-resolution (high frequency in z) or adaptive, object-dependent z-filtering. The non-adaptive solutions include z-filtering in the projection data domain with a fixed kernel (See K. Taguchi, U.S. Pat. No. 5,825,842 (1998), the contents of which are herein incorporated by reference), z-filtering in the image domain, and z-filtering in the projection data domain with a variable size of a kernel (See I. Zmora, U.S. Pat. No. 6,560,308, the contents of which are herein incorporated by reference).

One disadvantage of these solutions is that the z-resolution of the entire image is lost in a uniform fashion. Simply put, z-resolution is lost everywhere. Most of the causes of the windmill artifact, such as ribs, skulls, and spines, are located in the peripheral region of the images and the central region of images requires high spatial z-resolution.

The adaptive z-filtering includes filtering in the projection data domain (See J. Hsieh, “Adaptive Interpolation Approach for Multi-slice Helical CT Reconstruction,” Proc of SPIE Vol. 5032, pp. 1876-1833 (2003), the contents of which are herein incorporated by reference) and filtering in the image domain. Both methods change the kernel of the z-filtering, depending on object dependent indices, such as the gradient in z and require a significant amount of processing, which is undesirable.

### SUMMARY OF THE INVENTION

In light of the above-described difficulties, the Applicant developed the present invention. A first non-limiting aspect of the invention provides a method for obtaining data from a computed tomography (CT) scan, including: obtaining projection data from at least two detector rows in a CT system; filtering the projection data in a direction of the at least two detector rows to obtain filtered data in which windmill artifacts are reduced; and reconstructing image data from the filtered data.

Another aspect of the present invention provides an X-ray CT apparatus including: a helical scanning device configured to collect projection data while at least one of a gantry and a couch moves along an axial direction of the couch, the helical scanning device including, an X-ray source configured to generate X-rays, and a detector having detector elements arranged in at least two detector rows along the axial direction and configured to produce the projection data; and a processor including a filtering device configured to filter the projection data in a direction of the at least two detector rows to obtain filtered data in which windmill artifacts are reduced, and a reconstructing device configured to reconstruct the filtered data.

Yet another aspect of the present invention provides an X-ray CT apparatus including: a helical scanning device configured to collect projection data while at least one of a gantry and a couch moves along an axial direction of the couch, the helical scanning device including, an X-ray source configured to generate X-rays, and a detector having detector elements arranged in at least two detector rows along the axial direction and configured to produce the projection data; and a processor including means for filtering the projection data in a direction of the at least two detector rows to obtain filtered data in which windmill artifacts are reduced, and a reconstructing device configured to reconstruct the filtered data.

Another aspect of the present invention provides a computer program product storing instructions for execution on a computer system, which when executed by the computer system, causes the computer system to perform the following steps: obtaining projection data from at least two detector rows in a CT system; filtering the projection data in a direction of the at least two detector rows to obtain filtered data in which windmill artifacts are reduced; and reconstructing the filtered data.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1a illustrates a multi-slice detector where N=4;

FIG. 1b represents geometry of the present invention;

FIG. 2a illustrates an example of σ(r_{2D});

FIG. 2b illustrates a Gaussian distribution w(k);

FIGS. 3a-3c illustrate the definition of a distance;

FIG. 4 illustrates an example of w_{Gn}(0);

FIG. 5a illustrates an example of a function w_{z}^{L} used in both RF-soft and the RF-sharp algorithms;

FIG. 5b illustrates an example of a function w_{z}^{r} used in the RF-soft algorithm;

FIG. 6 illustrates pixels projected onto the detector; and

FIG. 7 illustrates the z-resolution index.

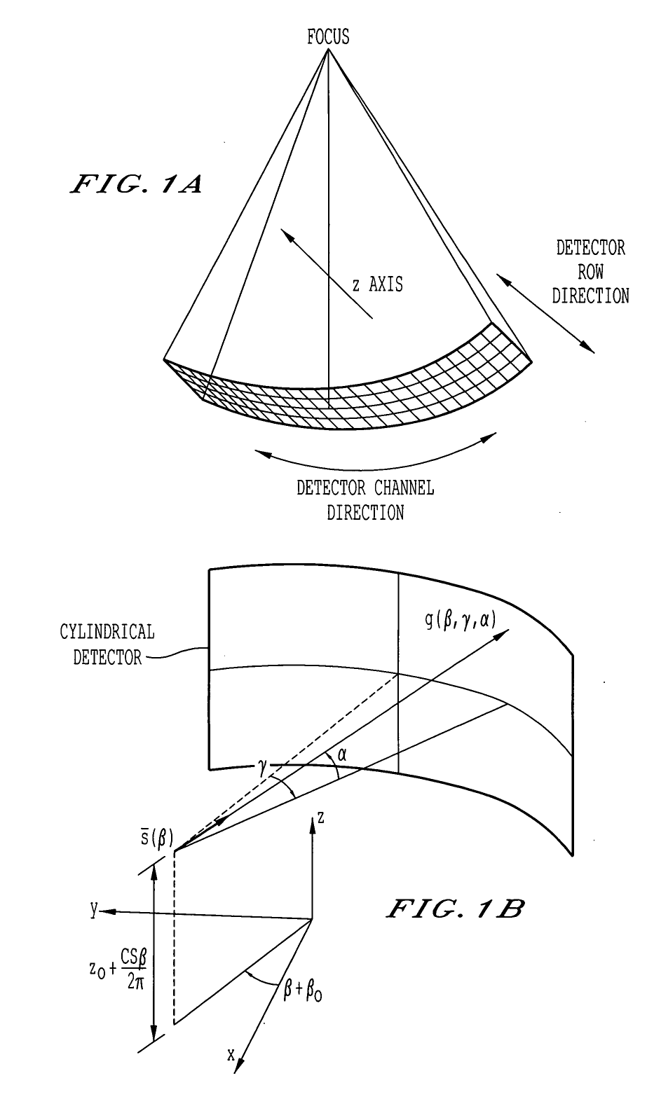

### DESCRIPTION OF THE PREFERRED EMBODIMENTS

The sharpness of the kernel increases with decreasing r_{2D} (e.g., a projected distance from the iso-center to the ray-sum onto the xy-plane), so that pixels near an iso-center have better z-resolution than in the periphery. Thus, the cause of the windmill artifact at the peripheral regions may be suppressed by applying a smoothing kernel to the corresponding detector channels.

To this end, filter data in a detector row direction may be obtained prior to image reconstruction. The kernel of the filter may be defined as a function of the ray-angle. That is, the kernel may be adjusted based on the projected distance from the iso-center to the ray-sum onto the xy-plane. Equation 1 (below) provides the desired function:

$${p}_{out} ( v , ch , row ) = {\sum }_{k = - K}^{K} [ {w}_{Gn} ( k , ch ) \cdot {p}_{in} ( v , ch , row + k ) ] \qquad ( 1 )$$

In Equation 1, pᵢₙ is the projection data, v represents the projection number index corresponding to the projection angle β, ch is the detector channel index corresponding to the ray angle γ, row is the detector row index corresponding to the cone angle α, w_{Gn} is the coefficient of the z-filter, and 2K−1 represents the size of the kernel.

Equations 2-4, below, represent the kernel:

$$\begin{matrix}{w}_{Gn} ( k , ch ) = w ( k , ch ) / {\sum }_{i = - K}^{K} w ( i , ch ) & ( 2 ) \\ w ( k , ch ) = 1 / \sqrt{2 π} \times {ⅇ}^{- \frac{1}{2} {( \frac{k}{σ ( {r}_{2 D} ( ch ) )} )}^{2}} - K \leq k \leq K , & ( 3 ) \\ {r}_{2 D} ( ch ) = R sin γ ( ch ) = R sin ( \frac{2 {γ}_{m} \cdot ( Cch - ch )}{Nch} ) & ( 4 )\end{matrix}$$

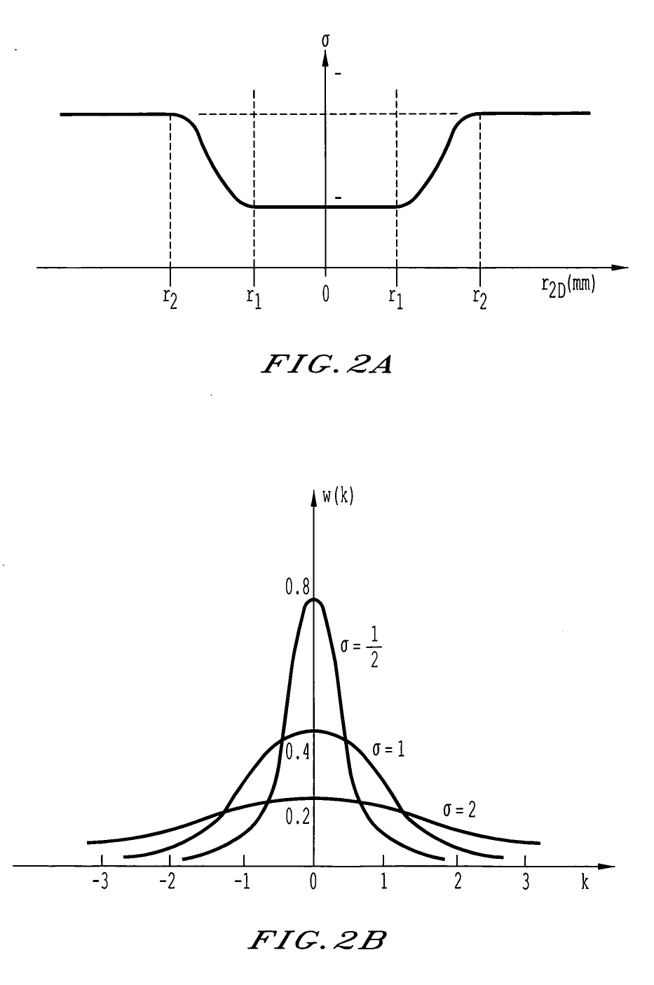

In Equations 2-4, R represents a distance from the focus to the iso-center, γₘ represents the maximum ray-angle, Nch represents the number of detector channels in one row, Cch is the detector channel index corresponding to γ=0, r_{2D} refers to the projected distance from the iso-center to the ray-sum onto the xy-plane, and σ(r_{2D}) defines a sharpness of the kernel. FIG. 2a illustrates an example of σ(r_{2D}), and FIG. 2b illustrates w(k) using a different σ.

Through this method, the sharpness of the kernel increases with a decreasing r_{2D}, so that pixels near the iso-center achieve a better z-resolution than in the periphery. Thus, the windmill artifact at the peripheral regions is suppressed by applying the smoothing kernel to the corresponding detector channels (e.g., by smoothing filtering).

The filtered projection data are then used for image reconstruction. Image reconstruction techniques include, but are not limited to a filtered backprojection and Fast Fourier Transform (FFT). According to the present invention, the γ-filtering may occur before the smoothing kernel is applied. γ-filtering may include applying a convolution process along a detector channel for image reconstruction such as ramp-filtering, but is not limited thereto.

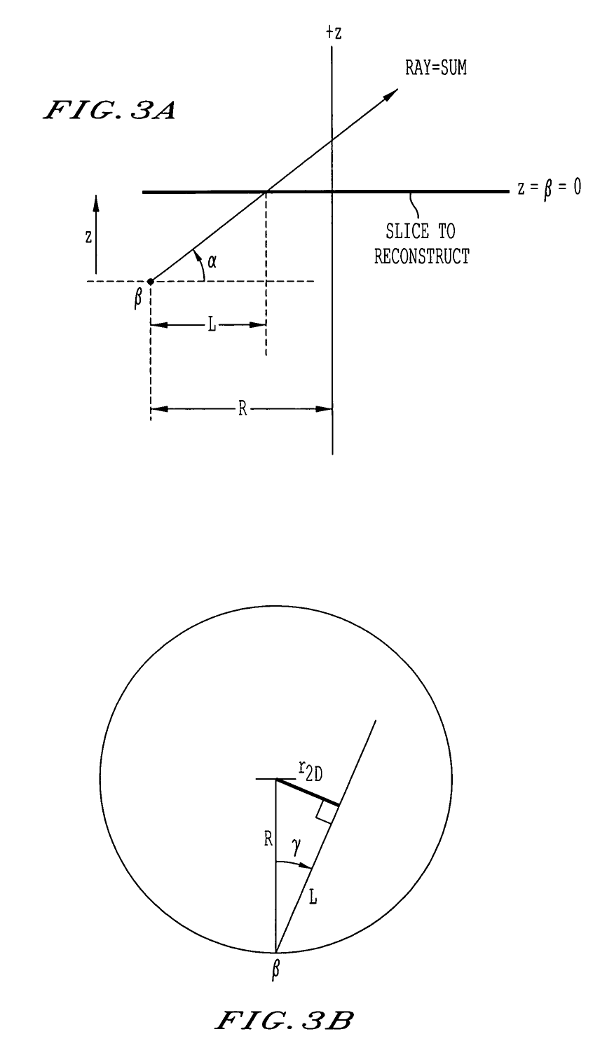

FIG. 3a shows the side view of a ray-sum, the rotation axis (z-axis), and the image to reconstruct (the voxel/pixel to backproject the datum/ray-sum). FIG. 3b illustrates the two-dimensional distance, r_{2D}, the projected distance from the iso-center to the ray-sum onto the xy-plane. FIG. 3c illustrates the three-dimensional distance, r_{3D}, the distance from the iso-center to the voxel on the ray-sum where the ray-sum coincides with the xy-plane to reconstruct (particular z location).

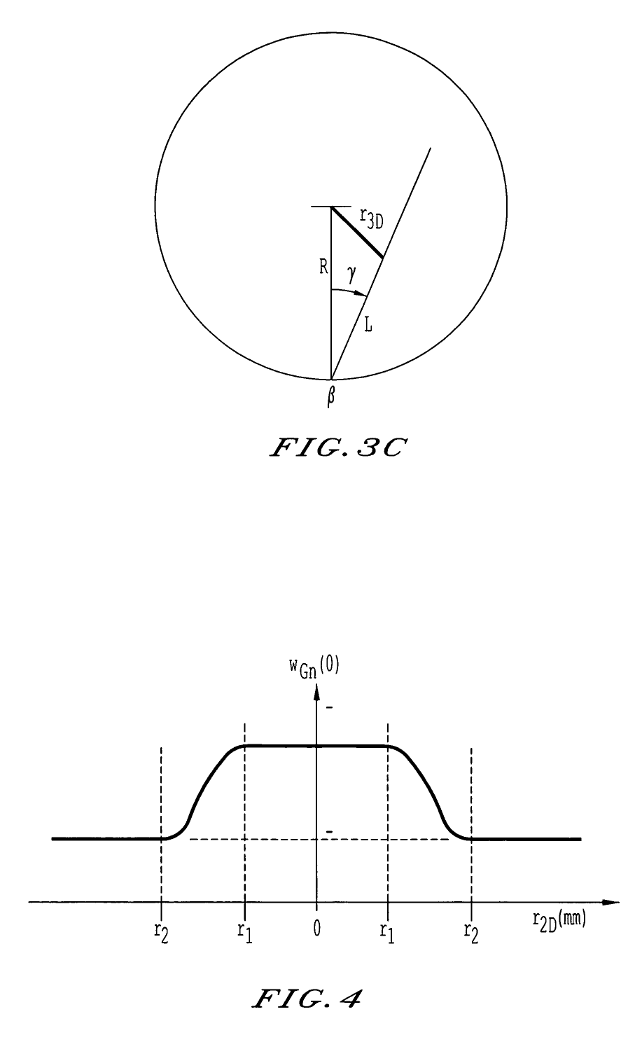

As a non-limiting alternative, Equation 5 (below) represents a case where the size of the kernel (2K+1) is fixed.
w_{Gn}(k,ch)|_{k≠0}=(1−w_{Gn}(0,ch))/(2K) (5)
An example of w_{Gn}(0) is represented in FIG. 4.

As a non-limiting alternative, it is possible to apply the filtering process in the detector row direction in the frequency domain rather than in the spatial domain. To this end, an FFT may be applied in the detector row direction, a channel-by-channel. The data having been transformed by the FFT may then be multiplied by a frequency representative of the z-filtering kernel. Finally, an inverse FFT may be applied to the filtered data.

Additionally, it is possible to apply a two-dimensional FFT by combining γ-filtering with row filtering according to the present invention. After the two-dimensional FFT is applied to the detector data, ramp-filtering may be applied in the γ-direction while smoothing filtering is applied in the row direction.

As a non-limiting alternative embodiment, it is possible to filter data in a detector row direction prior to image reconstruction. The kernel of the filter may be changed, based on the distance from the iso-center to the pixel corresponding to the detector cell. The following Equation 6 represents the filtering equation:

$${p}_{out} ( v , ch , row ) = {\sum }_{k = - K}^{K} [ {w}_{Gn} ( k , {r}_{3 D} ) \cdot {p}_{in} ( v , ch , row + k ) ] \qquad ( 6 )$$

Equations 7-16, shown below, describe the kernel:

$$\begin{matrix}{w}_{Gn} ( k , {r}_{3 D} ) = w ( k , {r}_{3 D} ) / {\sum }_{i = - K}^{K} w ( i , {r}_{3 D} ) & ( 7 ) \\ \begin{matrix}w ( k , {r}_{3 D} ) = 1 \sqrt{2 π} \times {ⅇ}^{- \frac{1}{2} {( \frac{k}{σ ( {r}_{3 D} )} )}^{2}} & - K \leq k \leq K ,\end{matrix} & ( 8 ) \\ σ ( {r}_{3 D} ) = \sqrt{{r}_{3 D} / {r}_{0}} & ( 9 ) \\ {r}_{3 D} = ( {r}_{3 D}^{′} - {r}_{0} ) \cdot {w}_{z}^{r} + {r}_{0} & ( 10 ) \\ {r}_{3 D}^{′} = ( \sqrt{{x}^{2} + {y}^{2}} ) = \sqrt{{L}^{2} - 2 RL \vert cos γ \vert + {R}^{2}} & ( 11 ) \\ γ ( ch ) = \frac{2 {γ}_{m} \cdot ( Cch - ch )}{Nch} & ( 12 ) \\ {L}_{β , α} = ( {L}_{β , α}^{′} - R ) \cdot {w}_{z}^{L} + R & ( 13 ) \\ {L}_{β , α}^{′} = \{ \begin{matrix}\vert {L}_{β , α}^{″} \vert & R - {r}_{m} < \vert {L}_{β , α}^{″} \vert < R + {r}_{m} \\ R - {r}_{m} & \vert {L}_{β , α}^{″} \vert < R - {r}_{m} \\ R + {r}_{m} & R + {r}_{m} < \vert {L}_{β , α}^{″} \vert\end{matrix} & ( 14 ) \\ {L}_{β , α}^{″} = z / tan α = \frac{R \cdot z}{d \cdot ( row - {row}_{c} )} & ( 15 ) \\ z = - CS \cdot β / 2 π = - CS \cdot ( v \cdot {v}_{c} ) / {N}_{vRev} & ( 16 )\end{matrix}$$

In Equations 7-16, row_{c} refers to the detector row index at α=0, d is the projected detector cell height to the iso-center, v_{c} denotes the projection number index at β=0 (e.g., when the focus is in the plane to be reconstructed), CS is the table feed per rotation (helical pitch), N_{vRev} is the number of projections per rotation.

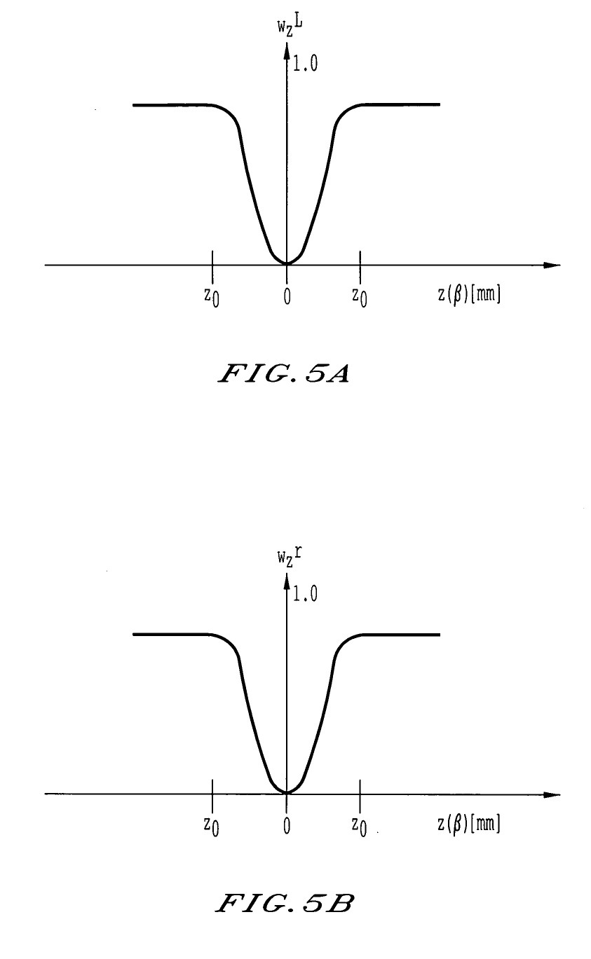

Two non-limiting variations are also possible when β is small: RF-sharp and RF-soft, where “RF” represents “Radius-Dependent Filtering Scheme.” Using the RF-sharp algorithm, w_{z}^{r} is fixed at 1.0. The w_{z}^{L} is a function of z(β), as illustrated in FIG. 5a. Equations 17 and 18 represent the RF-sharp algorithm.
If w_{z}^{L}→1, then L→L(γ,β,α); r→r(α,β,α); w_{Gn}→w_{Gn}(γ,β,α). (17)
Else if w_{z}^{L}→0, then L→R (fixed for all); r→r(γ); w_{Gn}→w_{Gn}(γ). (18)

The RF-soft algorithm is a case where both w_{z}^{L} and w_{z}^{r} are a function of z(β) as illustrated in FIGS. 5a-5b. Specifically, FIG. 5a illustrates an example of a function w_{z}^{L} used in both the RF-soft and RF-sharp algorithms. FIG. 5b illustrates an example of a function w_{z}^{r} used in the RF-soft algorithm. Equations 19 and 20, below, represent the RF-soft algorithm.
If w_{z}^{r}→1, then r→r(γ,β,α); w_{Gn}→w_{Gn}(γ,β,α). (19)
Else if w_{z}^{L}→0, then r→r₀(fixed for all); w_{Gn}→w_{Gn}(γ=γ(r₀))(fixed for all). (20)

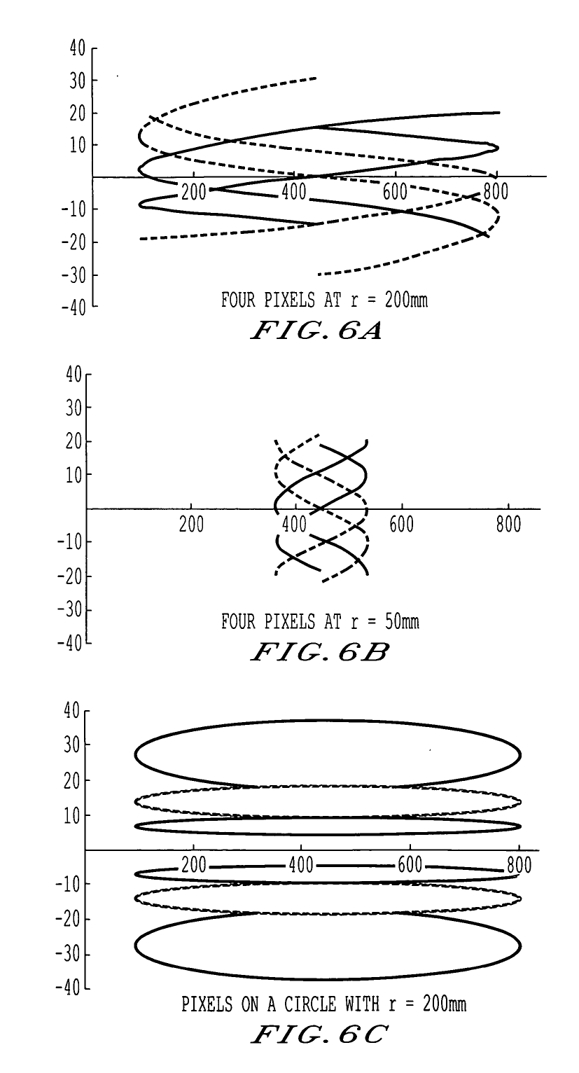

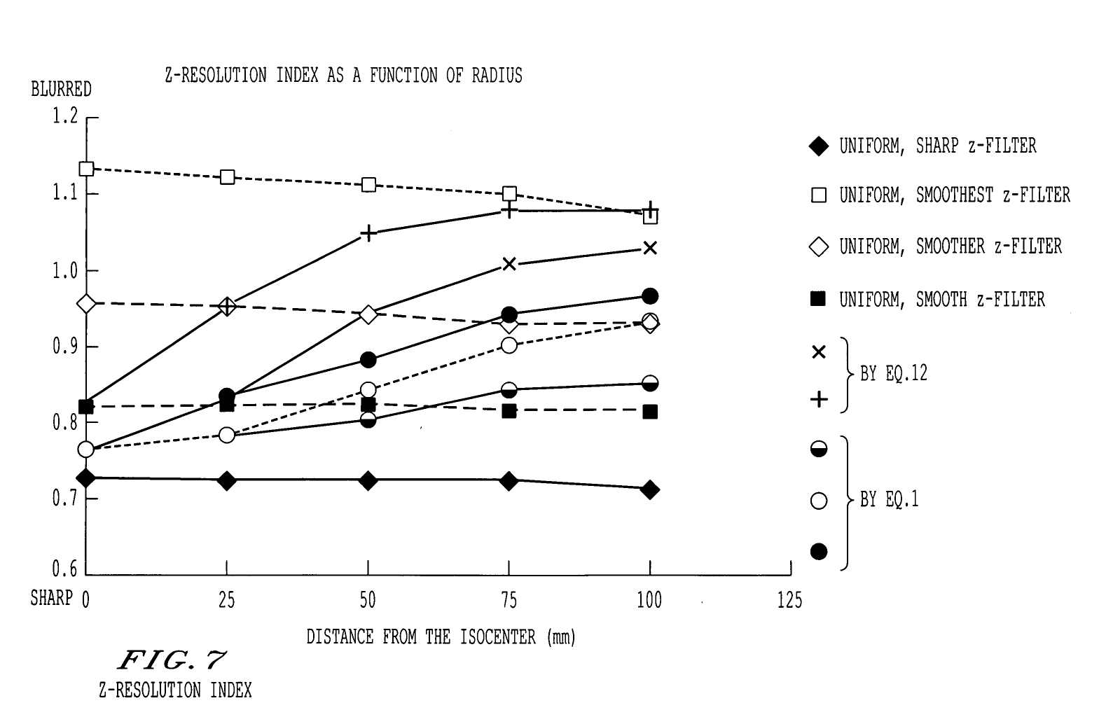

FIG. 6 illustrates the relationship between a detector cell and pixels to be reconstructed. The relationship illustrated in FIG. 6 led the present inventors to the concept of r_{3D}. In FIG. 6, the pixels were projected on the detector for β=[−π,π]. The horizontal axis represents the detector channel, and the vertical axis represents the detector row. As illustrated in FIG. 6, the projected region changes with β. FIG. 7 illustrates the z-resolution index using the RF-sharp algorithm and the RF-soft algorithm.

All embodiments of the present invention conveniently may be implemented using a conventional general purpose computer or micro-processor programmed according to the teachings of the present invention, as will be apparent to those skilled in the computer art. Appropriate software may readily be prepared by programmers of ordinary skill based on the teachings of the present disclosure, as will be apparent to those skilled in the software art.

As disclosed in cross-referenced U.S. Pat. No. 6,236,051, a computer may implement the methods of the present invention, wherein the computer housing houses a motherboard which contains a CPU, memory (e.g., DRAM, ROM, EPROM, EEPROM, SRAM, SDRAM, and Flash RAM), and other optional special purpose logic devices (e.g., ASICS) or configurable logic devices (e.g., GAL and reprogrammable FPGA). The computer also includes plural input devices, (e.g., keyboard and mouse), and a display card for controlling a monitor. Additionally, the computer may include a floppy disk drive; other removable media devices (e.g. compact disc, tape, and removable magneto-optical media); and a hard disk or other fixed high density media drives, connected using an appropriate device bus (e.g., a SCSI bus, an Enhanced IDE bus, or an Ultra DMA bus). The computer may also include a compact disc reader, a compact disc reader/writer unit, or a compact disc jukebox, which may be connected to the same device bus or to another device bus.

Examples of computer readable media associated with the present invention include compact discs, hard disks, floppy disks, tape, magneto-optical disks, PROMs (e.g., EPROM, EEPROM, Flash EPROM), DRAM, SRAM, SDRAM, etc. Stored on any one or on a combination of these computer readable media, the present invention includes software for controlling both the hardware of the computer and for enabling the computer to interact with a human user. Such software may include, but is not limited to, device drivers, operating systems and user applications, such as development tools. Computer program products of the present invention include any computer readable medium which stores computer program instructions (e.g., computer code devices) which when executed by a computer causes the computer to perform the method of the present invention. The computer code devices of the present invention may be any interpretable or executable code mechanism, including but not limited to, scripts, interpreters, dynamic link libraries, Java classes, and complete executable programs. Moreover, parts of the processing of the present invention may be distributed (e.g., between (1) multiple CPUs or (2) at least one CPU and at least one configurable logic device) for better performance, reliability, and/or cost. For example, an outline or image may be selected on a first computer and sent to a second computer for remote diagnosis.

The present invention may also be complemented with additional filtering techniques and tools to account for image contrast, degree of irregularity, texture features, etc.

The invention may also be implemented by the preparation of application specific integrated circuits or by interconnecting an appropriate network of conventional component circuits, as will be readily apparent to those skilled in the art.

The source of image data to the present invention may be any appropriate image acquisition device such as an X-ray machine, CT apparatus, and MRI apparatus. Further, the acquired data may be digitized if not already in digital form. Alternatively, the source of image data being obtained and processed may be a memory storing data produced by an image acquisition device, and the memory may be local or remote, in which case a data communication network, such as PACS (Picture Archiving Computer System), may be used to access the image data for processing according to the present invention.

Numerous modifications and variations of the present invention are possible in light of the above teachings. It is therefore to be understood that within the scope of the appended claims, the invention may be practiced otherwise than as specifically described herein.

1. A method for obtaining data from a computed tomography (CT) scan, comprising:
obtaining projection data from at least two detector rows in a CT system;
filtering the projection data in a direction of the at least two detector rows to obtain filtered data in which windmill artifacts are reduced; and
reconstructing image data from the filtered data.

2. The method according to claim 1, wherein the filtering is performed in relation to at least one of a ray angle and a distance from an iso-center to a detector cell.

3. The method according to claim 1, wherein the filtering is performed in relation to a distance from an iso-center to a voxel on a ray-sum where the ray-sum coincides with an xy-plane.

4. The method according to claim 1, wherein the reconstructing includes backprojecting.

5. The method according to claim 4, wherein the backprojecting includes at least one of applying Feldkamp reconstruction, advanced single-slice rebinning, applying an FFT, applying an inverse FFT, and adaptive multiple plane reconstruction.

6. An X-ray CT apparatus, comprising:
a helical scanning device configured to collect projection data while at least one of a gantry and a couch moves along an axial direction of the couch, the helical scanning device including,
an X-ray source configured to generate X-rays, and
a detector having detector elements arranged in at least two detector rows along the axial direction and configured to produce the projection data; and
a processor comprising,
a filtering device configured to filter the projection data in a direction of the at least two detector rows to obtain filtered data in which windmill artifacts are reduced, and
a reconstructing device configured to reconstruct the filtered data.

7. The X-ray CT apparatus according to claim 6, wherein the filtering device is configured to filter the projection data based on at least one of a ray angle and a distance from an iso-center to a detector cell.

8. The X-ray CT apparatus according to claim 6, wherein the filtering is performed in relation to a distance from an iso-center to a voxel on a ray-sum where the ray-sum coincides with an xy-plane.

9. The X-ray CT apparatus according to claim 6, wherein the reconstructing device includes a backprojecting device.

10. The X-ray CT apparatus according to claim 9, wherein the backprojecting device is configured to backproject the filtered data by applying at least one of Feldkamp reconstruction, advanced single-slice rebinning, applying an FFT, applying an inverse FFT, and adaptive multiple plane reconstruction.

11. An X-ray CT apparatus, comprising:
a helical scanning device configured to collect projection data while at least one of a gantry and a couch moves along an axial direction of the couch, the helical scanning device including,
an X-ray source configured to generate X-rays, and
a detector having detector elements arranged in at least two detector rows along the axial direction and configured to produce the projection data; and
a processor comprising,
means for filtering the projection data in a direction of the at least two detector rows to obtain filtered data in which windmill artifacts are reduced, and
a reconstructing device configured to reconstruct the filtered data.

12. The X-ray CT apparatus according to claim 11, wherein the means for filtering filters the projection data based on at least one of a ray angle and a distance from an iso-center to a detector cell.

13. The X-ray CT apparatus according to claim 11, wherein the filtering is performed in relation to a distance from an iso-center to a voxel on a ray-sum where the ray-sum coincides with an xy-plane.

14. The X-ray CT apparatus according to claim 11, wherein the reconstructing device includes a backprojecting device.

15. The X-ray CT apparatus according to claim 14, wherein the backprojecting device is configured to backproject the filtered data by applying at least one of Feldkamp reconstruction, advanced single-slice rebinning, applying an FFT, applying an inverse FFT, and adaptive multiple plane reconstruction.

16. A computer program product storing instructions for execution on a computer system, which when executed by the computer system, causes the computer system to perform the following steps:
obtaining projection data from at least two detector rows in a CT system;
filtering the projection data in a direction of the at least two detector rows to obtain filtered data in which windmill artifacts are reduced; and
reconstructing the filtered data.

17. The computer program product according to claim 16, wherein the filtering is performed in relation to at least one of a ray angle and a distance from an iso-center to a detector cell.

18. The computer program product according to claim 16, wherein the filtering is performed in relation to a distance from an iso-center to a voxel on a ray-sum where the ray-sum coincides with an xy-plane.

19. The computer program product according to claim 16, wherein the reconstructing includes backprojecting.

20. The computer program product according to claim 19, wherein the backprojecting includes at least one of applying Feldkamp reconstruction, advanced single-slice rebinning, applying an FFT, applying an inverse FFT, and adaptive multiple plane reconstruction.
